(19) United States
(12) Patent Application Publication
(10) Pub. No.: US 2007/0135747 A1
(43) Pub. Date: Jun. 14, 2007

(54) Toe glove device

(76) Inventor: Wen-Chieh Chan, Fongshan (TW)

Correspondence Address: G. LINK CO. LTD., 3550 BELL ROAD, MINOOKA, IL, 60447, US

(21) Appl. No.: 11/299,201

(22) Filed: Dec. 12, 2005

(51) Int. Cl. A61F 5/00

(52) U.S. Cl. 602/30

(57) ABSTRACT

A toe glove device includes a main toe glove, a secondary toe glove and a spacing pad. The main toe glove is snugly inserted with a toe. The secondary toe glove is disposed next to the main toe glove and snugly inserted with another toe next to the first toe. The spacing cushion pad is provided between the main toe glove and the secondary toe glove. Each of the toe gloves has an outer layer and a gel layer and the gel layer is attached to the inner side of the outer layer. The spacing cushion pad is an elastic gel pad. The hurt toe is inserted into the main glove and another toe next to the hurt toe is inserted into the secondary toe glove. The spacing cushion pad has a function of locating the main glove and the secondary glove and the hurt toe is capable of being corrected without phenomenon of toe overlapping.

### BACKGROUND OF THE INVENTION

1. Field of the Invention

The present invention is related to a toe glove device and particularly to a toe correcting glove device, which is capable of being inserted with toes of a user, for protecting the toes and avoiding the toes abnormal bending and overlapping each other.

2. Brief Description of the Related Art

The illness of toe becoming revered and distorted usually has the abnormal bent toe and even occurs overlapping toes. The illness is caused by the foot being bound a long period of time to result in dislocation of the toe joint in addition to the genetic factor. Clinically, the symptom of the illness is bulged at the location of toe's bone. The patient with toe becoming reversed and distorted is subjected to extreme inconvenience and trouble. In order to overcome the symptom, correctors can be used except choosing medical operation. One of the correctors shown in FIG. 1 provides a corrector 10, which has a toe glove 11 at an end thereof for fixing the toe and a binding band 12 at another end thereof to create a pull force. When the corrector 10 is pulled, the toe glove 11 creates a transverse force for correcting the toe head and avoiding phenomenon of overlapping toes. However, the corrector 10 is a medical curing appliance used in a specific period of curing time before recovery and the patient is unable to wear the corrector 10 with the shoes during the ordinary daily life. Thus, the corrector 10 has its limitations in use and effect.

### SUMMARY OF THE INVENTION

An object of the present invention is to provide a toe glove device which is capable being used even if the shoes are put on for protecting the hurt toe and avoiding abnormal toe bend and toe overlapping.

### BRIEF DESCRIPTION OF THE DRAWINGS

The detail structure, the applied principle, the function and the effectiveness of the present invention can be more fully understood with reference to the following description and accompanying drawings, in which:

FIG. 1 is a perspective illustrating the conventional corrector in use;

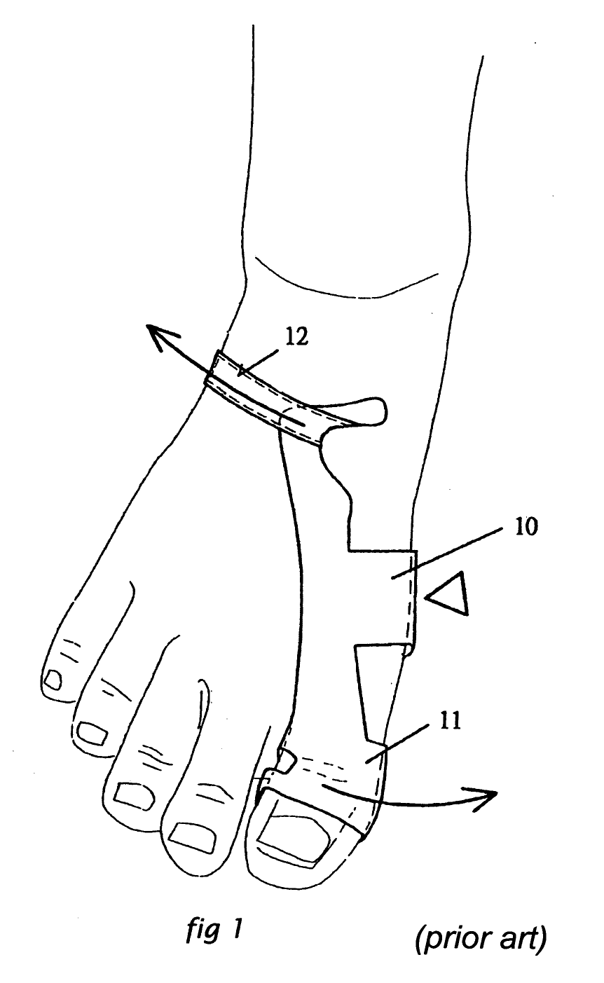

FIG. 2 is a perspective view of a preferred embodiment of a toe glove device according to the present invention;

FIG. 3 is a plan view of a toe glove shown in FIG. 2;

FIG. 4 is a sectional view along 4-4 line shown in FIG. 3;

FIG. 5 is a plan view illustrating the toe glove being in use;

FIG. 6 is a perspective view of another embodiment of the toe glove device according to the present invention; and

FIG. 7 is a plan view illustrating a bottom pad being joined to the toe glove device.

### DETAILED DESCRIPTION OF THE INVENTION

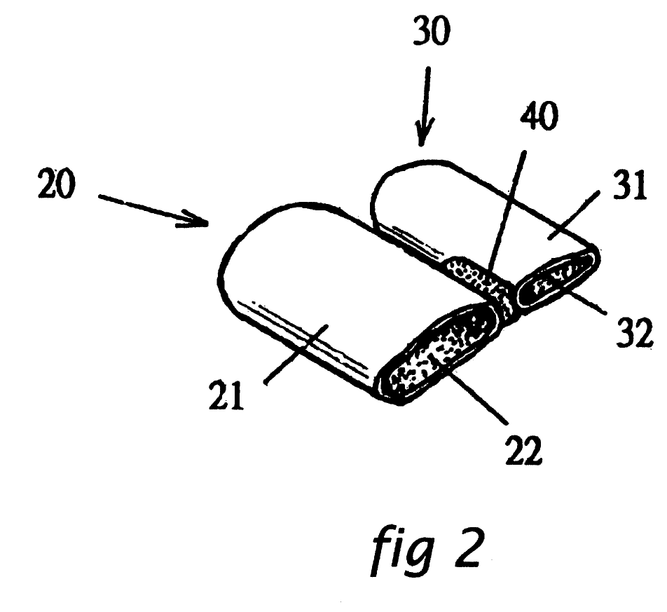

Referring to FIG. 2, a first embodiment of a toe glove device according to the present invention includes a main toe glove 20, a secondary toe glove 30 and a spacing cushion pad 40 between the main toe glove 20 and the secondary toe glove 30.

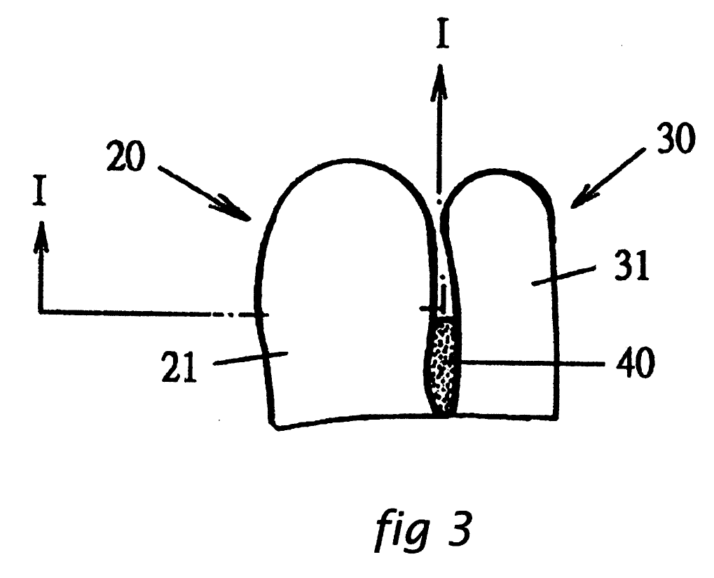

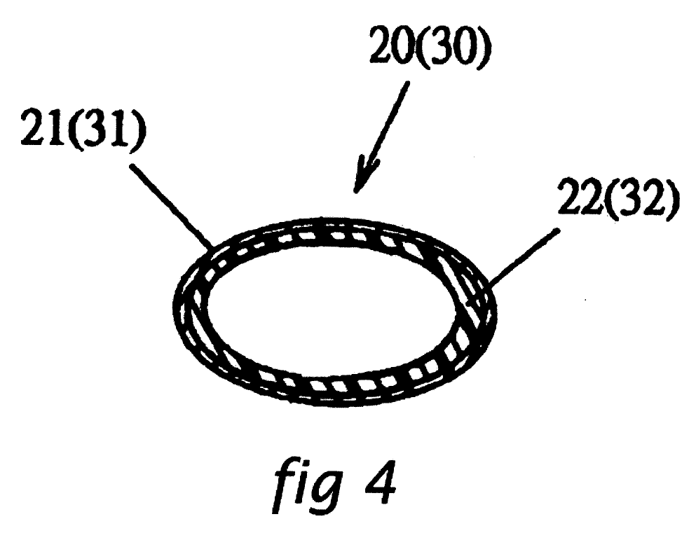

Referring to FIGS. 3 and 4 and companying with FIG. 2, the main toe glove 20, which is capable of being inserted with a toe of the user, provides an outer layer 21 made of cloth and an elastic gel layer 22 attached to the inner side of the outer layer 21.

Referring to FIGS. 3 and 4 and companying with FIG. 2 again, the secondary toe glove 30, which is disposed next to the main toe glove 20 and capable of being inserted with another toe of the user, provides an outer layer 31 made of cloth and an elastic gel layer 32 attached to the inner side of the outer layer 31.

Further, referring to FIGS. 3 and 4 in company with FIG. 2, the spacing cushion pad 40, which is provided beside either the main toe glove 20 or the secondary toe glove 30, is made of elastic gel.

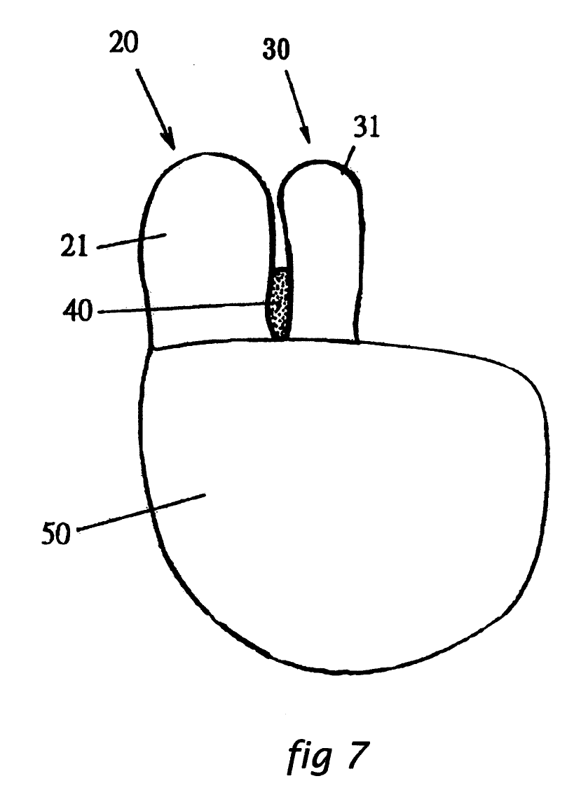

Referring to FIG. 7, a bottom pad 50 is joined to the front end of the main toe glove 20 and provides a shape of foot palm for offering softness and comfort while the foot palm stamping downward.

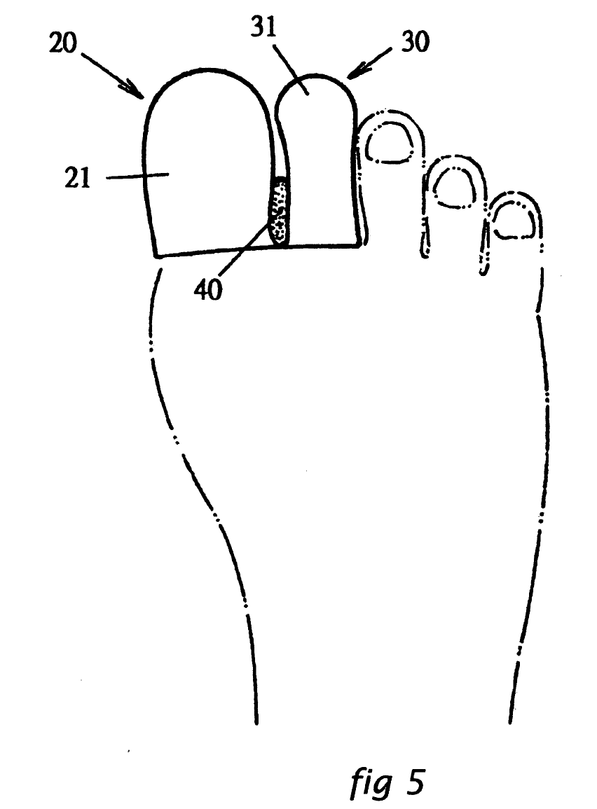

Referring to FIG. 5, when the toe glove device of the present invention is in use, the main glove 20 is inserted with the hurt toe and the hurt toe shown in the figure is the thumb toe. It is noted that the hurt toe can be another toe except the thumb toe. The secondary toe glove 30 is inserted with another toe next to the hurt toe. The spacing cushion pad 40 is disposed at the seam between the two toes after the main toe glove 20 and the secondary toe glove 30 fitting with the two toes. Hence, the spacing cushion pad 40 has a function of locating the main glove 20 and the secondary glove 30 and the main glove 20 is elastically separated from the secondary glove 30 with the spacing cushion pad 40 to prevent from overlapping toes. Further, the main toe glove 20 and the secondary toe glove 30 provide a gel layer 22, 32 at the inner sides thereof respectively for covering the toes snugly so that discomfort created by the locating pressure can be soothed or even avoided. Besides, socks and shoes can be put onto the feet after the toe glove device is used such that the hurt toe of the user can be corrected with the toe glove device of the present invention during the time of wearing the shoes for enhancing the effect of correction of hurt toe.

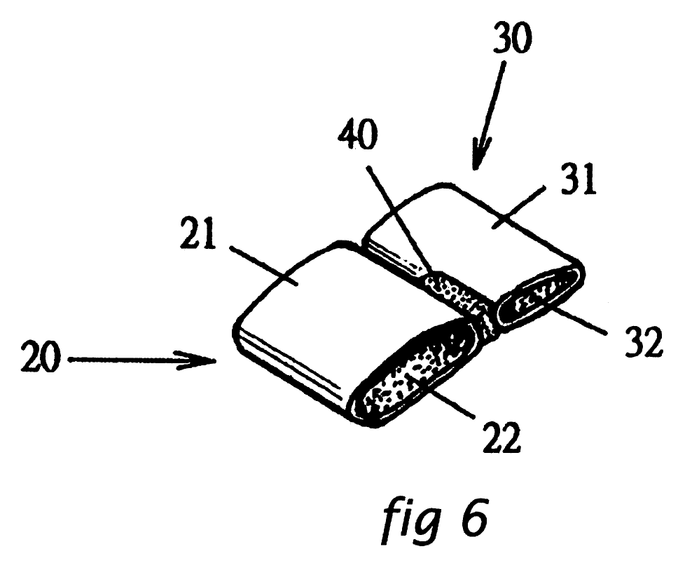

Referring to FIG. 6, the second embodiment of the toe glove device according to the present invention is illustrated. The difference of the present embodiment is in that the main toe glove 20 and the secondary toe glove 30 is uncovered at the front ends thereof for exposing the toes with the same effect as well.

It is appreciated that the toe glove device of the present invention is capable of protecting the hurt toe and preventing the hurt toe from bending for avoiding a phenomenon of toe overlapping. These are advantages unable to reach with the prior art.

While the invention has been described with referencing to preferred embodiments thereof, it is to be understood that modifications or variations may be easily made without departing from the spirit of this invention, which is defined by the appended claims.

What is claimed is:

1. A toe glove device, comprising
a main toe glove, being snugly inserted with a first toe;
a secondary toe glove, being disposed next to the main toe glove and snugly inserted with a second toe next to the first toe; and
a spacing cushion pad, being provided between the main toe glove and the secondary toe glove;
wherein, each of the toe gloves has an outer layer and a gel layer, which is attached to the inner side of the outer layer; and the spacing cushion pad is an elastic gel pad.

2. The toe glove device as defined in claim 1, wherein the toe gloves have a closed front end or an open front end respectively.

3. The toe glove device as defined in claim 1, wherein a front foot palm half shaped bottom pad is joined to the rear ends of the toe gloves with softness and comfort during the foot palm stepping forward.
